(19) United States
(12) Patent Application Publication
(10) Pub. No.: US 2011/0104301 A1
(43) Pub. Date: May 5, 2011

(54) METHODS OF REDUCING PAIN AND INFLAMMATION

(75) Inventors: Gerard Ahern, Alexandria, VA (US); Jose A. Matta, Woodbridge, VA (US)

(73) Assignee: Georgetown University, A Congressionally Chartered Institution of Higher Education

(21) Appl. No.: 12/995,264

(22) PCT Filed: May 29, 2009
(86) PCT No.: PCT/US09/45695

§ 371 Date: Nov. 30, 2010

Related U.S. Application Data
(60) Provisional application No. 61/130,379, filed on May 30, 2008.
(60) Provisional application No. 61/074,431, filed on Jun. 20, 2008.

(51) Int. Cl. A61K 33/24; A61K 31/35; A61K 31/125; A61K 31/66; A61K 31/05; A61K 31/352; A61K 31/7088; A61K 38/17; A61P 25/00

(52) U.S. Cl. 424/617; 514/453; 514/692; 514/102; 514/729; 514/454; 514/44 A; 514/18.3

(57) ABSTRACT

Provided herein are methods of treating or preventing pain and/or inflammation in a subject comprising administering to the subject a transient receptor potential (TRP) ion channel inhibitor.

### STATEMENT REGARDING FEDERALLY SPONSORED RESEARCH

This invention was made with government support under Grant No. 1R01NS055023-01A2 awarded by the National Institutes of Health. The government has certain rights in the invention.

### BACKGROUND

General anesthetics (GAs) are a diverse group of chemicals with the shared ability to suppress CNS activity and induce reversible unconsciousness. This immensely useful pharmacological property permits the ˜100 million surgeries performed worldwide each year. The molecular mechanisms of anesthesia have been extensively studied and there is now considerable evidence that GAs can inhibit CNS activity by discrete actions on membrane ion channels, in particular, through the activation of γ-aminobutyric acid (GABA) receptors. Strikingly, and in contrast to their inhibitory effects in the CNS, some GAs can stimulate peripheral nociceptors. For example, the i.v. anesthetics propofol and etomidate elicit “burning” pain on injection. Further, inhalation of volatile GAs (VGAs) can excite Aδ- and C-fiber neurons innervating the rabbit cornea, monkey skin, and canine airways. Indeed, neurogenic respiratory irritation limits the use of the more pungent anesthetics as induction agents. These excitatory effects of GAs on sensory nerves may explain, in part, why subanesthetic concentrations of these agents are hyperalgesic in rodents and in humans. Of particular clinical relevance, the administration of GAs coincides with surgically induced tissue damage, and the combination of nociceptor activation/sensitization and tissue injury has important implications for postsurgical pain and inflammation. Despite the potential importance of these effects, the underlying mechanisms and consequences of anesthetics activating nociceptors are yet to be determined.

### SUMMARY

Provided herein are methods of treating or preventing pain and/or inflammation in a subject comprising administering to the subject a transient receptor potential (TRP) ion channel inhibitor.

The details of one or more aspects are set forth in the accompanying drawings and description below. Other features, objects, and advantages will be apparent from the description and drawings, and from the claims.

### DESCRIPTION OF DRAWINGS

FIGS. 1A-1E show VGAs activate TRPA1. FIG. 1A shows representative current traces during application of isoflurane (0.9 mM, 2.9 MAC) in HEK293 cells expressing TRPM8, TRPV 1, or TRPA1. Positive responses were elicited by menthol (1 mM), capsaicin (1 μM), or AITC (100 μM). FIG. 1B shows isoflurane (0.9 mM) evoked inward currents in AITC-sensitive sensory neurons (n=11). FIG. 1C shows isoflurane activates TRPA1 in a dose-dependent manner with an EC50 of 180±20 μM (n=4-7) and a Hill coefficient of 1.6±0.2. At 2.7 mM isoflurane the response is reduced reflecting an additional blocking mechanism. (Inset) Example of washout of isoflurane; scale bars: 100 pA and 5 s. FIG. 1D shows isoflurane (0.25 mM) and desflurane (0.9 mM) activate single TRPA1 channels in outside-out patches from HEK293 cells (no activity was observed in mock-transfected cells). The Vₘ was +50 mV. All-points histogram from 2-s data segments are shown on the right. FIG. 1E shows the mean currents (fraction of isoflurane) evoked by 0.9 mM concentrations of halothane, sevoflurane, and desflurane. Data are means from five to six experiments.

FIGS. 2A-2G show noxious i.v. GAs activate TRPA1. FIGS. 2A and 2B show that in HEK293 cells, propofol and etomidate (100 μM) selectively activate TRPA1 without affecting TRPM8 or TRPV1 currents (Vₘ=−50 mV, n=6-8). FIG. 2C shows an I-V relationship for responses to propofol and AITC (1 mM, n=7). FIG. 2D shows dose-dependent activation by propofol (0.3-300 μM, n=4-6). FIG. 2E shows propofol (100 μM) activates single TRPA1 channels in an outside-out patch (n=3, Vₘ=+40 mV). All-points histograms reveal a decrease in unitary conductance from 108 to 94 pS. FIGS. 2F and 2G show propofol (100 μM) evoked inward currents and depolarized AITC-sensitive DRG neurons (n=6). Currents were blocked by camphor (0.5 mM).

FIGS. 3A-3D show GAs excite DRG neurons via TRPA1. The left panels of FIGS. 3A and 3C are representative Ca²⁺ transients evoked by desflurane (1.5 mM, 3 MAC), propofol (100 μM), and AITC (1 mM) in DRG neurons obtained from wild-type mice. The right panels of FIGS. 3A and 3C show the percentage of neurons responsive to desflurane (n=123), propofol (n=63), and AITC. The left panels of FIGS. 3B and 3D are representative Ca²⁺ transients evoked by desflurane propofol and capsaicin (100 nM) in DRG neurons obtained from TRPA1-null mice. The right panels of FIGS. 3B and 3D show the number of DRG neurons responsive to desflurane (n=125), propofol (n=120), or capsaicin. FIGS. 4A-4F show volatile anesthetics interact directly with TRPA1 channels. FIGS. 4A and 4B show activation of TRPA1 by hexanol (3 mM), octanol (1 mM), and decanol (0.6 mM) (n=4). FIG. 4C shows octanol (1.8 mM) and isoflurane (0.9 mM) modulate TRPA1 in a nonadditive fashion. FIG. 4D shows activation of TRPM8, TRPV1, and TRPA1 currents at −50 mV by isoflurane (0.9 mM) and octanol (1 mM), compared with maximal stimulation with menthol (1 mM), capsaicin (1 μM), and AITC (1 mM), respectively (n=4-6). FIG. 1E shows propofol (100 μM) and octanol (1.8 mM) produce an additive response at TRPA1. FIG. 1F shows the mean effects of octanol (1.8 mM) on isoflurane (0.9 mM) and propofol (100 μM)-evoked currents (n=5-6), *, P<0.01.

FIG. 5A-5D show TRPA1 mediates propofol-evoked, pain-related behavior. FIG. 5A shows topical application of propofol (50%) to the nasal epithelium evokes nocifensive behavior in wild-type (n=5) and TRPV1-null (n=4) mice. FIG. 5B shows propofol-induced nociception is abolished in TRPA1^{-/-} animals (n=5); *, P<1E-6 compared with TRPA1^{-/-} littermates (n=5). FIGS. 5C and 5D show integrated EMG activity from semitendinosus muscle of TRPA1^{+/-} and TRPA1^{-/-} mice after injection of 30 μA of propofol (500 μM) or capsaicin (50 μM, 5 min later) into the femoral artery (n=3 for both).

FIGS. 6A-6C show AITC-induced ear swelling is greater during anesthesia with isoflurane compared with sevoflurane. FIGS. 6A and 6B show AITC (0.6%, 20 μl) was applied to one ear of mice and the contralateral ear received mineral oil alone. Animals were anesthetized with 1.2 MAC (minimum alveolar concentration) of isoflurane or sevoflurane for 60 min followed by 60 min of recovery. Data show the change in ear thickness from baseline (both groups, n=7)*, P<0.01, AITC+Isoflurane versus other groups ANOVA; †, P<0.05 for isoflurane alone versus sevoflurane alone. FIG. 6C shows pungent (isoflurane and desflurane) but not smooth (methoxyflurane and sevoflurane) VGAs (0.5-0.65 mM) enhance currents evoked by AITC (10 μM) in TRPA1-expressing oocytes (n=3-4 for each point). *, P<0.05 versus AITC alone.

FIGS. 7A-7C show isoflurane enhances capsaicin-evoked TRPV1 currents. FIG. 7A shows representative current trace from a voltage-clamped neuron treated sequentially with isoflurane (0.9 mM), capsaicin (30 nM) and isoflurane plus capsaicin. FIG. 7B, upper trace, shows continuous recording of capsaicin sensitive channels in an outside/out patch (holding potential of +50 mV) in the presence of capsaicin (30 nM) or capsaicin plus isoflurane (0.9 mM). Lower traces (i-iii), expanded sections of recording from indicated timepoints. FIG. 7C shows dose-response curves in oocytes for activation of TRPV1 by capsaicin with or without isoflurane (0.9 mM, n=3-7 for each data point). The smooth curves are fits to a Hill function yielding EC50 values of 1.64±0.12 μM and 0.81±0.04 μM for control and isoflurane respectively.

FIGS. 8A and 8B show isoflurane enhances the sensitivity of TRPV1 to protons. FIG. 8A shows current trace from a TRPV1-expressing oocyte treated with pH 5.5 and pH 5.5 plus isoflurane (0.9 mM) solutions. FIG. 8B shows dose-response curves in oocytes for activation of TRPV1 by protons in the absence or presence of isoflurane (0.9 mM, n=3-7 for each data point). The smooth curves are fits to a Hill function yielding pEC50 of 4.95±0.15 and 5.23±0.10 for control and isoflurane respectively. Isoflurane also increased the maximal response from 14.3% to 32.2% of 10 μM capsaicin (P<0.01).

FIGS. 9A-9D shows volatile GAs increase the sensitivity of TRPV1 to voltage and heat. FIG. 9A shows TRPV1 currents activated by a family of voltage steps (−90 to 210 mV) under control conditions and with isoflurane (0.9 mM). FIG. 9B shows plots of tail current versus voltage-prepulse for control and isoflurane. Smooth lines are best fits to a Boltzmann function yielding V_{1/2} values of 161.5±2.7 mV and 129.0±3.6 mV. FIG. 9C shows current versus temperature plots in TRPV1-expressing oocytes for control (black), 0.5 mM (blue), or 0.9 mM (red) isoflurane. Currents are normalized to the maximum current evoked at 47° C. FIG. 9D shows mean thresholds of heat activation for control, 0.5 mM isoflurane, 0.9 mM isoflurane, PDBu (200 nM, 3 minutes) and PDBu+isoflurane (0.9 mm), *P<0.01 compared with control, or versus PDBu alone. Data are mean of 4-5 oocytes.

FIG. 10 shows isoflurane modulates TRPV1 at clinically-relevant concentrations. The mean fold increase in proton-evoked currents in oocytes produced by co-application with varying concentrations of isoflurane (0.1-2 mM, 0.3-3 MAC) or a second application of pH 5.5 alone. Data are the mean of 3-4 oocytes, *P<0.01 compared with pH 5.5 alone.

FIG. 11 shows modulation of TRPV1 by diverse volatile anesthetics. The relative potentiation of proton (pH5.5)-evoked currents by 0.6 mM concentrations of sevoflurane, isoflurane, enflurane and desflurane (n=3-4 for each data point). * P<0.01 compared with control. **P<0.01 between designated groups of VGAs (oneway ANOVA)

FIGS. 12A-12D show isoflurane activates TRPV1 in a PKC-dependent manner. FIG. 12A shows that pretreatment with PDBu (500 nM) halothane (0.9 mM) and isoflurane (0.9 mM) activates currents in TRPV1-expressing HEK293 cells. FIG. 12B shows AMG9810 (1 μM) inhibits the current evoked by isoflurane (0.9 mM) in a sensory neuron (pretreated with PDBu). FIG. 12C shows mean current evoked by isoflurane in TRPV1-expressing HEK293 cells and capsaicin-sensitive sensory neurons, with or without PDBu treatment. Data are normalized to responses evoked by a saturating capsaicin concentration (5 μM), and the number of cells are given in parentheses. FIG. 12 D shows single TRPV 1 channel activity in an outside-out patch from a sensory neuron in response to isoflurane (0.9 mM) and AMG9810 (1 μM). The holding potential was +60 mV.

FIGS. 13A-13D show isoflurane and bradykinin synergistically excite TRPV1 and sensory neurons. FIG. 13A shows bradykinin (BK, 10 μM) enhances capsaicin (30 nM)-evoked currents in sensory neurons. FIGS. 13B and 13C show co-application of BK and isoflurane (0.9 mM) induces inward currents in sensory neurons and these currents are inhibited by capsazepine (1 μM). FIG. 13D shows co-application of BK and isoflurane depolarizes a capsaicin-sensitive sensory neuron under current clamp. The arrow indicates −60 mV.

FIGS. 14A-14E show volatile anesthetics interact directly with TRP channels. FIG. 14A shows representative TRPV 1 current traces in response to voltage steps in the presence of various alcohols. FIG. 14B shows Boltzmann fits to the conductance measured at the end of test potential. FIGS. 14C and 14D show summary of changes in TRPV1 V1/2 and maximal conductance induced by alcohols (n=4-7 cells). Concentrations of ethanol (508 mM), hexanol (3 mM), octanol (1 mM), decanol (0.6 mM) and dodecanol (0.1 mM) were chosen according to the solubility limitations of these alcohols as described previously (Peoples and Weight, PNAS 92:2825-9 (1995)). FIG. 14E shows octanol (1.8 mM) and isoflurane (0.9 mM) modulate TRPV1 in a non-additive fashion.

FIG. 15 is a schematic showing a model of synergistic activation of TRPs by anesthetics and inflammatory mediators in sensory nerves. Tissue injury leads to accumulation of inflammatory mediators such as proteases and bradykinin which engage their respective G-protein coupled receptors (protease receptor, PAR; bradykinin receptor, BKR) expressed on sensory nerves. In turn, this leads to sensitization of TRPV1 and TRPA1 via phospholipase C dependent pathways. VGAs act directly on TRPs to further enhance their activity. Finally, depolarization and Ca²⁺ entry via TRPs evokes release of inflammatory peptides including substance P (SP) and calcitonin gene-related peptide (CGRP).

FIGS. 16A, 16B and 16C are graphs showing the responses of WT and chimeric mouse/drosophila TRPA1 channels to desflurane. FIG. 16A shows current-voltage relationship for a dTRPA1-expressing HEK293 cell in response to desflurane (1 mM) and a mTRPA1-expressing cell in response to desflurane and AITC (0.5 mM). Note that control currents were subtracted. FIGS. 16B and 16C show current-voltage plots for the chimeric proteins, dTRPA1-mN and mTRPA1-dTM5.

### DETAILED DESCRIPTION

Provided herein are methods of treating or preventing pain and/or inflammation in a subject comprising administering to the subject a transient receptor potential (TRP) ion channel inhibitor. Thus, provided is a method for reducing or preventing inflammation in a subject comprising administering to the subject an agent that inhibits the activity or expression a transient receptor potential (TRP) ion channel inhibitor. Also provided is a method for reducing or preventing pain in a subject comprising administering to the subject an agent that inhibits the activity or expression a TRP ion channel inhibitor. Optionally, the method comprises selecting a subject in need of relief of pain or inflammation. Optionally, the subject is under anesthesia. Optionally, the method further comprises selecting a subject under anesthesia. Optionally, the TRP is transient receptor potential vanilloid (TRPV 1) and TRP ankyrin (TRPA1). Optionally, the inhibitor binds the TM5 domain of TRPA1. Optionally, the inhibitor binds SEQ ID NO:1 or SEQ ID NO:2. Optionally, the pain and/or inflammation is associated with administration of an anesthetic to the subject. Optionally, the subject is a surgical patient. Optionally, the pain is post-surgical pain. Optionally, the TRP inhibitor is administered at the same time, before or after an anesthetic is administered to the subject.

As used herein, a transient receptor potential (TRP) ion channel refers to transient receptor potential vanilloid (TRPV) and TRP ankyrin (TRPA) and homologs, variants and isoforms thereof. There are a variety of sequences that are disclosed on Genbank, at www.pubmed.gov, and these sequences and others are herein incorporated by reference in their entireties as well as for individual subsequences contained therein. For example, the amino acid and nucleic acid sequences of human TRPA1 can be found at GenBank Accession Nos. NP_{—}015628.2 and NM_{—}007332.2, respectively. The amino acid and nucleic acid sequences of human TRPV 1 can be found at GenBank Accession Nos. NP_{—}542436.2 and NM_{—}080705.3, respectively.

Provided herein are TRP inhibitors for the treatment or prevention of pain and inflammation Inhibitors of TRP include, but are not limited to, inhibitory peptides, small molecules, drugs, functional nucleic acids and antibodies.

Inhibitors of TRP include inhibitory peptides or polypeptides. As used herein, the term peptide, polypeptide, protein or peptide portion is used broadly herein to mean two or more amino acids linked by a peptide bond. Protein, peptide and polypeptide are also used herein interchangeably to refer to amino acid sequences. The term fragment is used herein to refer to a portion of a full-length polypeptide or protein. It should be recognized that the term polypeptide is not used herein to suggest a particular size or number of amino acids comprising the molecule and that a peptide of the invention can contain up to several amino acid residues or more. Peptides can be tested for their ability to inhibit TRP by methods known to those of skill in the art, such as, for example, phage display and yeast two-hybrid assays. Inhibitory peptides also include dominant negative mutants of TRP. Dominant negative mutations (also called antimorphic mutations) have an altered phenotype that acts antagonistically to the wild-type or normal protein. Thus, dominant negative mutants of TRP act to inhibit the normal TRP protein. Such mutants can be generated, for example, by site directed mutagenesis or random mutagenesis. Proteins with a dominant negative phenotype can be screened for using methods known to those of skill in the art, for example, by phage display. Such peptides are selected based on their ability to inhibit TRP.

Nucleic acids that encode the aforementioned peptide sequences are also disclosed. These sequences include all degenerate sequences related to a specific protein sequence, i.e., all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. A wide variety of expression systems may be used to produce peptides as well as fragments, isoforms, and variants.

Suitable TRPA1 inhibitors include those described in WO/2008/013861, which is incorporated herein by reference in its entirety. Other TRPA1 specific inhibitors include those described in Chen et al., Journal of Biomolecular Screening, Vol. 12, No. 1, 61-69 (2007), which is incorporated herein by reference in its entirety. TRPV1 specific inhibitors are described in Bruce R. Bianchi, Robert B. Moreland, Connie R. Faltynek, Jun Chen. ASSAY and Drug Development Technologies. Jun. 1, 2007, 5(3): 417-424. Other TRP inhibitors include, but are not limited to, wortmannin, camphor, phosphatidylinositol-4,5-bisphosphate (PIP2), high levels of menthol, AP18, cannabinoids such as WIN 55, 212-2, HC-030031, gadolinium, ruthenium red, capsazepine, AMG 517, SB366791, Iodo-resiniferatoxin, resiniferatoxin, LJO-328, and SC0030. See Karashima et al., “Modulation of the transient receptor potential channel TRPA1 by phosphatidylinositol 4,5-biphosphate manipulators,” Pflügers Archiv European Journal of Physiology (2008); Kim et al., “Inhibition of Transient Receptor Potential A1 by Phosphatidylinositol-4,5-bisphosphate,” Am J Physiol Cell Physiol. (2008); Karashima, et al., The Journal of Neuroscience, 2007, 27(37):9874-9884; Patwardhan et al., PNAS, 2006, vol. 103, no. 30, 11393-11398; Taylor-Clarke et al., Mol. Pharmacol. 2008, 73(2):274-81; WO/2008/013861; Bang et al., European Journal of Neuroscience, Volume 26 Issue 9 Page 2516-2523, 2007; Gunthorpe and Szallasi, Curr Pharm Des. 2008; 14(1):32-41; Anderson et al., Thorax. 2004; 59: 730-731; Johansen et al., Toxicological Sciences 89(1):278-86 (2006); and Veronesi et al., Toxicol Sci. 2006 January; 89(1):1-3, which are incorporated herein by reference in their entireties.

Also provided herein are functional nucleic acids that inhibit expression of TRPA1 and TRPV1. Such functional nucleic acids include but are not limited to antisense molecules, aptamers, ribozymes, triplex forming molecules, RNA interference (RNAi), and external guide sequences. Thus, for example, a small interfering RNA (siRNA) could be used to reduce or eliminate expression of TRP. Functional nucleic acids are nucleic acid molecules that have a specific function, such as binding a target molecule or catalyzing a specific reaction. Thus, for example, a small interfering RNA (siRNA) could be used to reduce or eliminate expression of TRP. Examples of siRNA molecules that inhibit TRP are described in Obata et al., J. Clin. Invest. 115(9): 2393-2401 (2005), which is incorporated herein by reference in its entirety.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Representative examples of how to make and use aptamers to bind a variety of different target molecules can be found in, for example, U.S. Pat. Nos. 5,476,766 and 6,051,698.

Ribozymes are nucleic acid molecules that are capable of catalyzing a chemical reaction, either intramolecularly or intermolecularly. There are a number of different types of ribozymes that catalyze nuclease or nucleic acid polymerase type reactions which are based on ribozymes found in natural systems, such as hammerhead ribozymes, hairpin ribozymes and tetrahymena ribozymes). There are also a number of ribozymes that are not found in natural systems, but which have been engineered to catalyze specific reactions de novo (for example, but not limited to U.S. Pat. Nos. 5,807,718, and 5,910,408). Ribozymes may cleave RNA or DNA substrates. Representative examples of how to make and use ribozymes to catalyze a variety of different reactions can be found in U.S. Pat. Nos. 5,837,855; 5,877,022; 5,972,704; 5,989,906; and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. Representative examples of how to make and use triplex forming molecules to bind a variety of different target molecules can be found in U.S. Pat. Nos. 5,650,316; 5,683,874; 5,693,773; 5,834,185; 5,869,246; 5,874,566; and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. Representative examples of how to make and use EGS molecules to facilitate cleavage of a variety of different target molecules be found in U.S. Pat. Nos. 5,168,053; 5,624,824; 5,683,873; 5,728,521; 5,869,248; and 5,877,162.

Gene expression can also be effectively silenced in a highly specific manner through RNA interference (RNAi). Short Interfering RNA (siRNA) is a double-stranded RNA that can induce sequence-specific post-transcriptional gene silencing, thereby decreasing or even inhibiting gene expression. In one example, an siRNA triggers the specific degradation of homologous RNA molecules, such as mRNAs, within the region of sequence identity between both the siRNA and the target RNA. Sequence specific gene silencing can be achieved in mammalian cells using synthetic, short double-stranded RNAs that mimic the siRNAs produced by the enzyme dicer. siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion's SILENCER® siRNA Construction Kit (Ambion, Austin, Tex.).

Proteins that inhibit TRP, such as TRPA1 or TRPV1, also include antibodies with antagonistic or inhibitory properties. In addition to intact immunoglobulin molecules, fragments, chimeras, or polymers of immunoglobulin molecules are also useful in the methods taught herein, as long as they are chosen for their ability to inhibit TRP. Optionally, the antibody binds the TM5 domain of TRPA1. The antibodies can be tested for their desired activity using in vitro assays, or by analogous methods, after which their in vivo therapeutic or prophylactic activities are tested according to known clinical testing methods.

The term antibody is used herein in a broad sense and includes both polyclonal and monoclonal antibodies. Monoclonal antibodies can be made using any procedure that produces monoclonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro. The monoclonal antibodies may also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

As used throughout, antibody fragments include Fv, Fab, Fab′ or other antigen binding portion of an antibody. Digestion of antibodies to produce fragments thereof, e.g., Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross linking antigen.

The antibody fragments, whether attached to other sequences, also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term antibody or antibodies can also refer to a human antibody and/or a humanized antibody. Examples of techniques for human monoclonal antibody production include those described by Cole et al. (Monoclonal Antibodies and Cancer Therapy, Alan R. Liss, p. 77, 1985) and by Boerner et al. (J. Immunol., 147(1): 86 95, 1991). Human antibodies (and fragments thereof) can also be produced using phage display libraries (Hoogenboom et al., J. Mol. Biol., 227:381, 1991; Marks et al., J. Mol. Biol., 222:581, 1991). The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551 255 (1993); Jakobovits et al., Nature, 362:255 258 (1993); Bruggermann et al., Year in Immunol., 7:33 (1993)). Specifically, the homozygous deletion of the antibody heavy chain joining region (J(H)) gene in these chimeric and germ line mutant mice results in complete inhibition of endogenous antibody production, and the successful transfer of the human germ line antibody gene array into such germ line mutant mice results in the production of human antibodies upon antigen challenge.

Antibody humanization techniques generally involve the use of recombinant DNA technology to manipulate the DNA sequence encoding one or more polypeptide chains of an antibody molecule. Accordingly, a humanized form of a non human antibody (or a fragment thereof) is a chimeric antibody or antibody chain that contains a portion of an antigen binding site from a non-human (donor) antibody integrated into the framework of a human (recipient) antibody. Fragments of humanized antibodies are also useful in the methods taught herein. Methods for humanizing non human antibodies are well known in the art. For example, humanized antibodies can be generated according to the methods of Winter and co workers (Jones et al., Nature, 321:522 525 (1986), Riechmann et al., Nature, 332:323 327 (1988), Verhoeyen et al., Science, 239:1534 1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

Methods of screening for agents that inhibit the activity of TRP are provided. Such a screening method comprises the steps of providing a cell that expresses a TRP or a fragment of a TRP (for example, TRPA1 or TRPV1, or a fragment thereof), contacting the cell with a candidate agent to be tested and determining whether the candidate agent prevents the expression or activation of TRP. Optionally, the cell expresses the TM5 domain of TRPA1. Optionally, the cell expresses SEQ ID NO:1 or SEQ ID NO:2. Another method of screening for agents that inhibit the activity of TRP comprises the steps of providing a sample comprising TRP or a fragment of a TRP, contacting the sample with a candidate agent to be tested and determining whether the candidate agent prevents the activation of TRP. Optionally, the sample comprises the TM5 domain of TRPA1. Optionally, the sample comprises SEQ ID NO:1 or SEQ ID NO:2. The provided cells that express TRP or a fragment of the TRP can be made by infecting the cell with a virus comprising TRP or a fragment of TRP wherein the TRP or fragment thereof is expressed in the cell following infection. The cell can also be a prokaryotic or an eukaryotic cell that has been transfected with a nucleotide sequence encoding TRP or a variant or a fragment thereof, operably linked to a promoter. Using DNA recombination techniques well known by the one skill in the art, protein encoding DNA sequences can be inserted into an expression vector, downstream from a promoter sequence. Alternatively, the cell expressing TRP optionally naturally expresses TRP.

Such methods allow one skilled in the art to select candidate agents that inhibit TRP expression or activity. Such agents may be useful as active ingredients included in pharmaceutical compositions. Methods for determining whether the candidate agent prevents expression or activation of TRP are known. The assay can be, for example, a proteolytic assay or one of the provided methods described in the examples below.

Pharmaceutical compositions comprising one or more of the inhibitors or agents provided herein may include carriers, thickeners, diluents, buffers, preservatives, surface active agents and the like in addition to the molecule of choice. Pharmaceutical compositions may also include one or more active ingredients such as antimicrobial agent, a chemotherapeutic agent, and the like. The compositions of the present application can be administered in vivo in a pharmaceutically acceptable carrier. By pharmaceutically acceptable is meant a material that is not biologically or otherwise undesirable. Thus, the material may be administered to a subject, without causing undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The disclosed compositions can be administered in a number of ways depending on whether local or systemic treatment is desired, and on the area to be treated. Thus, the disclosed compositions can be administered, for example, orally, parenterally (e.g., intravenously), by intramuscular injection, by intraperitoneal injection, transdermally, extracorporeally, or topically.

The materials may be in solution or suspension (for example, incorporated into microparticles, liposomes, or cells). These may be targeted to a particular cell type via antibodies, receptors, or receptor ligands. Suitable carriers and their formulations are described in Remington: The Science and Practice of Pharmacy (21th ed.) ed. David B. Troy, Lippincott Williams & Wilkins, 2005. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8.5, and more preferably from about 7.8 to about 8.2. Further carriers include sustained release preparations such as semipermeable matrices of solid hydrophobic polymers, which matrices are in the form of shaped articles, e.g., films, liposomes or microparticles. Certain carriers may be more preferable depending upon, for instance, the route of administration and concentration of composition being administered.

The terms effective amount and effective dosage are used interchangeably. The term effective amount is defined as any amount necessary to produce a desired physiologic response. Effective amounts and schedules for administering the compositions may be determined empirically, and making such determinations is within the skill in the art. The dosage ranges for the administration of the compositions are those large enough to produce the desired effect in which the symptoms or disorder are affected. The dosage should not be so large as to cause substantial adverse side effects, such as unwanted cross-reactions, anaphylactic reactions, and the like. Generally, the dosage will vary with the age, condition, sex, type of disease and extent of the disease in the patient, route of administration, or whether other drugs are included in the regimen, and can be determined by one of skill in the art. The dosage can be adjusted by the individual physician in the event of any contraindications. Dosage can vary and can be administered in one or more dose administrations daily, for one or several days. Guidance can be found in the literature for appropriate dosages for given classes of pharmaceutical products.

The provided compositions can be administered in combination with one or more other therapeutic or prophylactic regimens. As used throughout, a therapeutic agent is a compound or composition effective in ameliorating a pathological condition. Illustrative examples of therapeutic agents include, but are not limited to, an anti-inflammatory agents and pain medications.

Anti-inflammatory agents that may be administered with the provided compositions include, but are not limited to, glucocorticoids and the nonsteroidal anti-inflammatories, aminoarylcarboxylic acid derivatives, arylacetic acid derivatives, arylbutyric acid derivatives, arylcarboxylic acids, arylpropionic acid derivatives, pyrazoles, pyrazolones, salicylic acid derivatives, thiazinecarboxamides, e-acetamidocaproic acid, S-adenosylmethionine, 3-amino-4-hydroxybutyric acid, amixetrine, bendazac, benzydamine, bucolome, difenpiramide, ditazol, emorfazone, guaiazulene, nabumetone, ninesulide, orgotein, oxaceprol, paranyline, perisoxal, pifoxime, proquazone, proxazole, and tenidap.

As used throughout, by a subject is meant an individual. Thus, the subject can include, for example, domesticated animals, such as cats and dogs, livestock (e.g., cattle, horses, pigs, sheep, and goats), laboratory animals (e.g., mice, rabbits, rats, and guinea pigs) mammals, non-human mammals, primates, non-human primates, rodents, birds, reptiles, amphibians, fish, and any other animal. The subject can be a mammal such as a primate or a human. The term subject also includes individuals of different ages. Thus, a subject includes an infant, child, teenager or adult.

As used herein the terms treatment, treat or treating refers to a method of reducing the effects of a disease or condition or symptom of the disease or condition. Thus in the disclosed method treatment can refer to a 10%, 20%, 30%, 40%, 50%, 60%, 70%, 80%, 90% or 100% reduction in the severity of an established disease or condition or symptom of the disease or condition. For example, a method for treating a disease is considered to be a treatment if there is a 10% reduction in one or more symptoms of the disease in a subject as compared to control. Thus the reduction can be a 10, 20, 30, 40, 50, 60, 70, 80, 90, 100% or any percent reduction in between 10 and 100 as compared to native or control levels. It is understood that treatment does not necessarily refer to a cure or complete ablation of the disease, condition or symptoms of the disease or condition.

As used herein, the terms prevent, preventing and prevention of a disease or disorder refers to an action, for example, administration of a therapeutic agent, that occurs before a subject begins to suffer from one or more symptoms of the disease or disorder, which inhibits or delays onset of the severity of one or more symptoms of the disease or disorder. As used herein, references to decreasing, reducing, or inhibiting include a change of 10, 20, 30, 40, 50, 60, 70, 80, 90 percent or greater as compared to a control level. Such terms can include but do not necessarily include complete elimination.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if an inhibitor is disclosed and discussed and a number of modifications that can be made to a number of molecules including the inhibitor are discussed, each and every combination and permutation of the inhibitor, and the modifications that are possible are specifically contemplated unless specifically indicated to the contrary. Likewise, any subset or combination of these is also specifically contemplated and disclosed. This concept applies to all aspects of this disclosure including, but not limited to, steps in methods of using the disclosed compositions. Thus, if there are a variety of additional steps that can be performed it is understood that each of these additional steps can be performed with any specific method steps or combination of method steps of the disclosed methods, and that each such combination or subset of combinations is specifically contemplated and should be considered disclosed.

Throughout this application, various publications are referenced. The disclosures of these publications in their entireties are hereby incorporated by reference into this application.

### EXAMPLES

### Example 1

### General Anesthetics Activate Transient Receptor Potential Ion Channel Vanilloid (TRPV1), Which Results in Enhanced Pain and Inflammation

### Materials and Methods

Electrophysiology. HEK 293F cells were transfected with rat TRPV1, TRPA1, and TRPM8 (gift of David Julius, University of California, San Francisco). Dorsal root ganglia were cultured from adult mice (C57B16/J wild type and TRPV1-null, and mixed B6/129 background TRPA1-null) in Neurobasal +2% B-27 medium (Invitrogen), 0.1% L-glutamine and 1% penicillin/streptomycin. Whole-cell and single-channel patch-clamp recordings were performed by using an EPC8 amplifier (HEKA Electronics). For whole-cell and excised patch recordings the bath solution contained 140 mMNaCl, 4 mMKCl, 1 mMMgCl₂, 1.2 mMCaCl₂, 10 mMHepes, 10 mM glucose, pH 7.3. For cell-attached experiments, NaCl was replaced with KCl in the bath solution. The pipette solution contained 140 mM NaCl or KCl, 10 mM Hepes, 5 mM EGTA, pH 7.3. For neuronal recording NaCl was replaced with K-gluconate (plus 1 mMATP, 0.2 mM GTP). Solutions were applied via a gravity-fed system. Separate outlets were used to apply capsaicin and AITC solutions to avoid contamination. Voltage-dependent properties were measured as described in Matt and Ahern, J. Physiol. 585:469-482 (2007). Current-voltage measurements comprised a 200-ms ramp from −150 mV to +200 mV. The baseline currents under control conditions were subtracted. For cell-attached experiments, peak amplitudes were measured from all-points histograms, and open probability was measured as NPₒ>750 ms. Defolliculated Xenopus laevis oocytes were injected with ≈10 ng of hTRPA1 (gift of Ardem Patapoutian, The Scripps Research Institute, La Jolla, Calif.). Oocytes were placed in a Perspex chamber and continuously superfused (5 ml min⁻¹) with Ca²⁺-free solution containing 100 mM NaCl, 2.5 mM KCl, 5 mM Hepes, 1 mM MgC12 and titrated to pH 7.3 with ˜5 mM NaOH.

Ca²⁺ Imaging. Neurons were loaded with 1 μM Fluo4-AM (Molecular Probes) for 20 min and washed for a further 10-20 min before recording. The dye was excited at 488±15 nm. Emitted fluorescence was filtered with a 535±25 nm bandpass filter, captured by a SPOT RT digital camera (Diagnostic Instruments) and read into a computer. Analysis was performed offline by using Simple PCI software (Compix Inc.).

Behavioral Experiments and Neurogenic Inflammation. Animal experiments were performed according to National Institutes of Health and institutional guidelines. Propofol (50% in mineral oil, 20 μl) was applied to the nasal epithelium of male C57/B16 and TRPV1-null mice and mixed B6/129 background TRPA1^{-/+} and TRPA1^{-/-} mice (5-7 weeks). Nocifensive behavior (nose wiping in sawdust bedding) was recorded for 2 min with a video camera and the duration was subsequently measured by a blinded observer. Application of capsaicin (10 mM) produced similar behavior in wild-type but not in TRPV 1-null mice, establishing that this is a bona fide nocifensive behavior. Electromyographic (EMG) activity was recorded via platinum electrodes from the semitendinosus muscle in mice anesthetized with urethane (1.3 g/kg) as described in Ando and Watanabe, Br J. Anaesth. 95:384-92 (2005). The EMG signal was recorded by using a low-pass cutoff frequency of 200 Hz and integrated offline by using a 100-ms time window. To induce the flexor reflex response, 30 μl of vehicle (0.01% DMSO), propofol (500 μM), or capsaicin (50 μM) were administered at a 5-min interval into the femoral artery via a PE10 catheter. Neurogenic inflammation was induced in male CBJ/A mice (4-6 weeks) with 20 μl of mustard oil (0.6%) applied to the front and back surface of one ear, and mineral oil was applied to the other (Inoue et al., Eur. J. Pharmacol. 333:231-240 (1997)). Animals were anesthetized with isoflurane or sevoflurane in oxygen by using anesthetic-specific vaporizers (Vapomatic); the concentrations in the chamber were maintained at ˜1.2 MAC confirmed with a gas analyzer (Ohmeda). Ear thickness was recorded by using an engineer's micrometer (Mitutoyo Corp.) before mustard-oil application and thereafter every 15 min for 60 min of anesthesia and 60 min of recovery.

Volatile General Anesthetics and Chemicals. Saturated stock solutions of volatile GAs were prepared in gas-tight bottles by dissolving excess anesthetic agents in bath solutions overnight. From these stock solutions fresh dilutions were made up every 40-60 min. Concentrations of GAs in the bath solutions were verified by using a modified head-space gas chromatography method. The equivalent MACs were calculated by using published conversion factors reported for halothane (0.27 mM), isoflurane (0.31 mM), desflurane (0.51 mM), sevoflurane (0.35 mM), and enflurane (0.64 mM) in rat at 37° C. (Franks and Lieb, Anesthesiology 84:716-20 (1996)). Alcohols with <6 carbons were dissolved directly into extracellular solution, and alcohols containing 6 carbons or more were dissolved in DMSO and then diluted into extracellular solutions that were sonicated for 20 min. All other drugs were prepared as stock solutions in DMSO or ethanol and diluted into physiological solution before experiments. Drug vehicles in final recording solutions were 0.05-0.1% DMSO or ethanol, concentrations with no tested biological effect at TRP channels used in this study.

Statistical Analysis. Data are given as mean±SEM. and statistical significance was evaluated by using ANOVA or Student's t test.

### Results

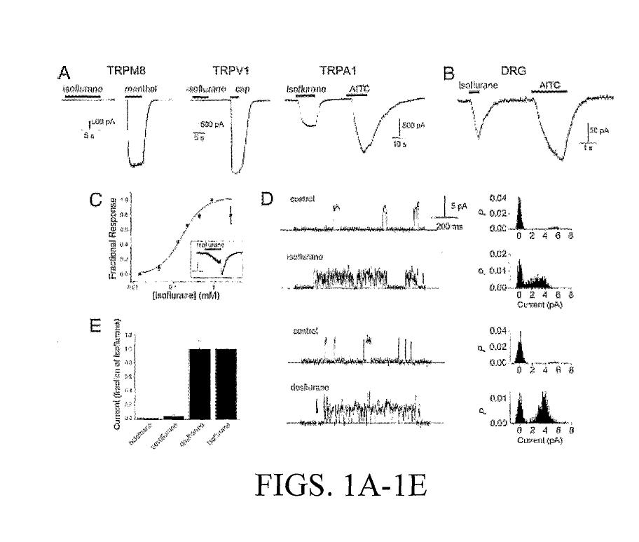

Noxious Volatile and Intravenous GAs Activate TRPA1. Several VGAs are known to stimulate nociceptors and it was determined whether this is mediated by TRP channels. Application of the pungent agent isoflurane [0.9 mM, or 2.9 minimum alveolar concentration (MAC)] produced inward currents in voltage-clamped, TRPA1-expressing HEK293 cells, but failed to activate TRPM8 and TRPV1 (FIG. 1A). Similarly, isoflurane (0.9 mM, 2.9 MAC) evoked currents in 11 of 35 (31%) neurons from wild-type mice tested under voltage-clamp, and 10 (91%) of these cells were also sensitive to AITC (FIG. 1B). Isoflurane activated TRPA1 in a dose-dependent manner (FIG. 1C), with an EC50 of 0.18±0.02 mM (0.57 MAC). Thus, these effects of isoflurane occur at relevant clinical concentrations (≈1-3 MAC).

Next, VGAs possessing differing pungencies were compared for activity at TRPA1. The pungent anesthetics, isoflurane and desflurane, robustly activated the channel, whereas the nonpungent agents sevoflurane and halothane were without effect (FIG. 1E). This relationship replicates the perceived pungency of VGAs when administered to patients (Eger, Int. Anesthesiol. Clin. 33:61-80 (1995)). These effects of isoflurane and desflurane were retained in cell-free patches from TRPA1-expressing HEK293 cells and AITC-sensitive neurons (FIG. 1D); both VGAs enhanced single-channel gating, but also reduced the single-channel conductance from ≈110 pS to ≈60 pS (0.23 mM isoflurane) and ≈80 pS (0.9 mM desflurane). This block was voltage-dependent and relieved at depolarized potentials. Thus, these agents (isoflurane, in particular) produce both agonistic and pore-blocking actions at TRPA1, and this explains the bimodal dose-response relationship that shows a peak at ≈1 mM and a reduction at higher concentrations of isoflurane (FIG. 1C).

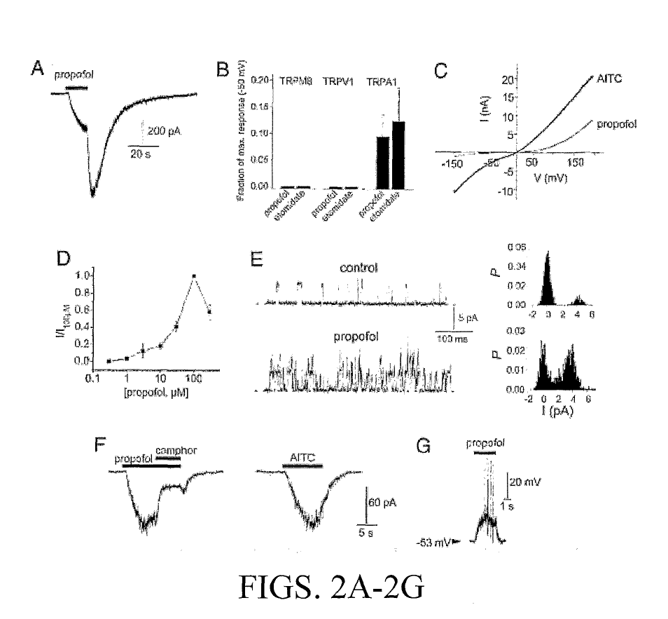

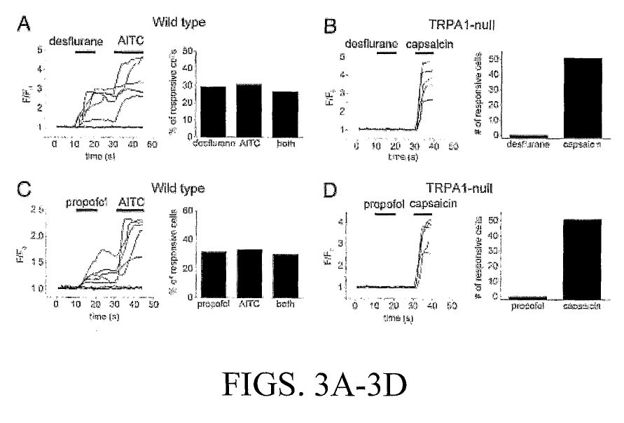

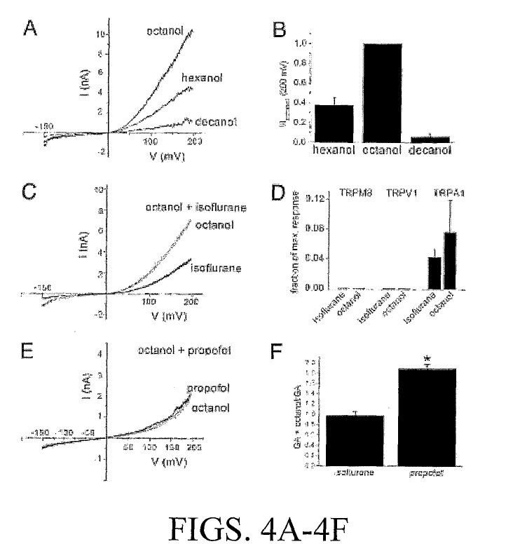

The i.v. GAs, propofol and etomidate, are associated with marked pain on injection. This pain occurs in 80% to 90% of patients; however, the underlying mechanisms are unknown. It was examined whether propofol and etomidate could excite sensory neurons through a direct modulation of TRP channels. In voltage-clamped HEK293 cells (membrane potential, −50 mV) both propofol and etomidate (100 μM) produced a robust activation of TRPA1 but were without effect on TRPV1 or TRPM8 channels (FIGS. 2 A and B). This activation occurred over the clinically relevant concentration range of 1-100 μM (FIG. 2D); the free concentration of propofol in clinical formulations is ≈100 μM (Doenicke et al., Anesth. Analg. 82:472-474 (1996)). On washout of propofol there was a surge in current suggesting an additional pore-blocking effect of the anesthetic (FIG. 2A). Accordingly, single-channel measurements showed that propofol both increased TRPA1 activity and reduced the unitary conductance (FIG. 2E). As with the inhalation agents this block was voltage-dependent (FIG. S1B). Thus, responses to propofol were greater at depolarized potentials, ≈8% and 38% of the full agonist, AITC, at −150 mV and +200 my, respectively (FIG. 2C). Propofol evoked inward currents were also observed in AITC-sensitive DRG neurons (n=6; FIG. 2F) and these currents were sensitive to a TRPA1 inhibitor, camphor. Furthermore, propofol depolarized these neurons under current clamp to elicit action potentials (FIG. 2G). To explore whether propofol could sensitize TRPV 1 and TRPM8, its effect on voltage-dependent activation was examined. Propofol and etomidate (100 μM) produced a small reduction in the half-maximal voltage (V_{1/2}) for TRPV1 activation of 10.5 and 9.3 mV, respectively (n=4-5). Propofol was without effect on TRPM8 (ΔV_{1/2}=1.7 mV, n=6), whereas etomidate increased the V_{1/2} by 25.5 mV (n=5). Thus, the predominant action of these GAs is to activate TRPA1, but etomidate can additionally block TRPM8. GAs Excite Sensory Nerves by Selectively Activating TRPA1. Next, to determine whether TRPA1 is the primary sensory nerve target for irritant GAs calcium imaging in DRG neurons was performed. FIG. 3A shows that desflurane (1.5 mM, 3 MAC) evoked a Ca²⁺ increase in a subset of neurons cultured from wild-type mice (36 of 123 cells). These desflurane-responsive cells were all sensitive to AITC. In contrast, no responses to desflurane were evident in cells obtained from TRPA1-null mice (FIG. 3B; n=125). Thus, TRPA1 appears to be essential for transducing the excitatory effect of VGAs in sensory neurons. Similar calcium-imaging analysis was performed with propofol. FIG. 3C shows that propofol selectively evoked a Ca²⁺ rise in AITC-sensitive neurons, with ≈30% of cells exhibiting dual sensitivity to propofol and AITC. In contrast, no responses to propofol were observed in neurons cultured from TRPA1-null mice (FIG. 3D; n=120). Furthermore, a total of 43% of these TRPA1-deficient cells were sensitive to capsaicin (FIGS. 3 B and D), thereby excluding a significant contribution of TRPV1 in desflurane and propofol signaling. Taken together, the data indicate that TRPA1 is a major determinant of the sensory nerve excitation produced by noxious GAs.

VGAs Directly Activate TRPA1. GAs could potentially modulate TRPA1 by modulating [Ca²⁺]ᵢ, or cellular signaling cascades. The presence of extracellular Ca²⁺ enhanced the response to GAs (FIG. S2); however, activation persisted when Ca²⁺ was removed (and with 5 mM intracellular EGTA), indicating a Ca²⁺-independent mechanism. Further, it was observed that both volatile and i.v. GAs effectively modulated TRPA1 in cell-free patches (FIGS. 1D and 2E) suggesting that these anesthetics signal in a membrane-delimited fashion, not via a soluble second messenger. Indeed, there is accumulating evidence that GAs can directly regulate ligand-gated ion channels. VGAs and alcohols share a common binding pocket in GABAA and glycine receptors, located between transmembrane domains 2 and 3 (20, 21).

Alcohol modulation of these receptors exhibits a carbon chain-length “cutoff”; the potency of alcohols increase with carbon chain length up until this cutoff, after which further increases in molecular size no longer increase alcohol potency (Mascia et al., PNAS 97:9305-9310 (2000); and Mihic, Nature 389:385-9 (1997)). These data are consistent with the existence of a cavity on these proteins that is accessible only to alcohols of a finite molecular volume. A similar cutoff with TRPA1 was observed. FIGS. 4 A and B shows that alcohols of 6-12 carbons enhanced activation of TRPA1 with a cutoff between octanol and decanol. Next, it was determined whether alcohols and VGAs act at similar binding site(s) on TRPA1. It was predicted that these chemicals would produce an additive response if they acted at different sites. In contrast, it was observed that isoflurane (0.9 mM) produced negligible effects on TRPA1 when applied together with an apparent saturating dose of octanol (1.8 mM) (FIGS. 4 C and F). Therefore, these data are consistent with VGAs and alcohols acting at a common site(s) (which reach saturation with submaximal efficacy). Note that this result cannot be explained by an overall “ceiling effect” on channel gating, because the responses to octanol were submaximal (<50% of 1 mMAITC at +200 mV). However, co-application of propofol and octanol produced an additive response at TRPA1 (FIGS. 4 E and F) suggesting that propofol acts at a distinct site from alcohols and VGAs. To further confirm a common action of alcohols and VGAs, the relative ability of these compounds to activate different TRPs was compared. FIG. 4D shows that at holding potential of −50 mV, octanol and isoflurane selectively activated TRPA1 with negligible effects at TRPV1 and TRPM8. Thus, octanol and isoflurane exhibit a similar activation profile at TRP channels, consistent with a common mechanism of action.

AITC and several other volatile compounds are electrophiles and can activate TRPA1 by covalent modification of N-terminal cysteines (Hinman et al., PNAS 103:19564-19568 (2006); Macpherson, Nature 445:541-5 (2007)). This does not seem to be the case for GAs because their chemical structures do not support such a mechanism. Moreover, in contrast to AITC, it was observed that successive applications of isoflurane could evoke TRPA1 currents (FIG. S3). However, isoflurane failed to activate TRPA1 after AITC treatment, suggesting that covalent modification renders TRPA1 unresponsive to GAs. AITC similarly depresses activation by voltage (Macpherson, Nature 445:541-5 (2007)) and menthol (Karashima, J. Neurosci. 27:9874-9884 (2007)), suggesting an allosteric mechanism of inhibition.

Finally, it was determined whether GAs activate by altering TRPA1 voltage sensitivity. To avoid the confound of GA-induced pore block, TRPA1 open probability in cell-attached patches was measured. Desflurane shifted the V_{1/2} from 72.1 to 42.5 mV and increased the maximal open probability >6-fold.

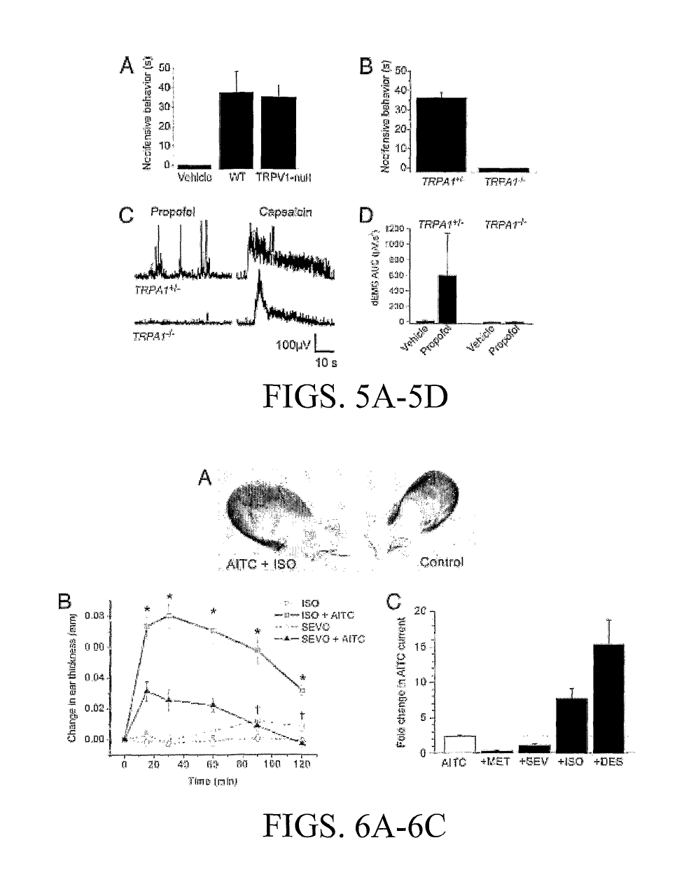

These observations suggest that, although voltage enhances the activation produced by desflurane, GAs can nonetheless act in a voltage-independent manner. TRPA1 Mediates Propofol-Induced Pain. Next, it was determined whether TRPA1 mediates the well described pain accompanying injections of propofol. First, it was determined whether topical application of propofol could induce nocifensive behaviors. FIG. 5A shows that when applied to the nasal epithelium, propofol induced ≈40 s of pain-related behavior (see Materials and Methods) over a 2-min period, whereas the vehicle (mineral oil) was without effect. A similar nocifensive response to propofol was seen in TRPV1-null mice (FIG. 5A). In contrast, nocifensive behavior was completely absent in TRPA1-null mice (FIG. 5B); whereas TRPA1^{+/-} littermates exhibited a robust response of ≈35 s. Second, the effects of propofol in a vascular-pain model were tested by using the flexor reflex response (Ando and Watanabe, Br. J. Anaesth. 95:384-392 (2005)). FIGS. 5 C and D shows that propofol, injected into the femoral artery, evoked reflex muscle activity in TRPA1^{+/-} mice but produced no responses in TRPA1-null animals. In contrast, capsaicin produced robust responses in both groups. Thus, taken together these data indicate that TRPA1 is critical for propofol-evoked nociception.

Isoflurane Evokes Greater Neurogenic Inflammation Compared with Sevoflurane. Excitation of sensory nerves can evoke the release of neuropeptides that contribute to inflammation. It was therefore determined whether VGAs could modulate this process through their actions at TRPA1. To test this, AITC was applied to the ears of mice—a commonly used model of neurogenic inflammation (Inoue et al., Eur. J. Pharmacol. 333:231-240 (1997))—and compared the ear swelling when animals were anesthetized either with isoflurane or sevoflurane (1.2 MAC, see Materials and Methods). FIGS. 6 A and B shows that AITC induced significantly greater swelling in animals anesthetized with isoflurane at all time points measured (15-120 min, n=7, P<0.01). Isoflurane also caused a small increase in swelling in the unpainted ear at 90 and 120 min. However, this effect did not occur in the absence of AITC when animals were administered isoflurane alone, suggesting that it was because of an interaction of isoflurane and AITC vapors in the chamber. These effects of isoflurane and sevoflurane on AITC-evoked inflammation paralleled the effect of these VGAs on AITC-evoked currents. FIG. 6C shows that the pungent agents isoflurane and desflurane markedly enhanced AITC-evoked currents in TRPA1-expressing oocytes, whereas sevoflurane and another nonpungent VGA, methoxyflurane, did not. Thus, the level of AITC-evoked inflammation during anesthesia correlates with the ability of VGAs to potentiate TRPA1. Taken together, these data suggest that VGAs, when administered in vivo, can differentially modulate TRPA1 to modulate neurogenic signaling.

### Example 2

### General Anesthetics Sensitize the Capsaicin Receptor Transient Receptor Potential Ion Channel Ankyrin (TRPA1)

### Materials and Methods

HEK cell and sensory neuron electrophysiology. HEK 293F cells (Invitrogen) were cultured in DMEM supplemented with 1% non-essential amino acids and 10% fetal calf serum. Cell cultures were maintained at 37° C. with 5% CO2. Cells were transfected with rat TRPV 1 (gift of David Julius), and GFP cDNA using Lipofectamine™ Transfection Reagent (Invitrogen) and used 24-48 h after transfection. Nodose ganglia were obtained from adult mice (C57B16/J and TRPV1-null), cut, digested with collagenase, and cultured in Neurobasal +2% B-27 medium (Invitrogen), 0.1% L-glutamine and 1% penicillin/streptomycin on poly-D lysine-coated glass coverslips at 37° C. in 5% CO₂. Neurons were used within 24-36 hr of culture. Whole-cell and single-channel patch clamp recordings were performed using an EPC8 amplifier (HEKA). The current signal was low-pass filtered at 1-3 kHz and sampled at 4 kHz. Currents were further filtered for display purposes. For whole-cell and excised patch recordings the bath solution contained (in mM): 140 NaCl, 4 KCl, 1 MgCl₂, 1 EGTA, 10 HEPES, 10 glucose pH 7.3 (290 mOsm). The pipette solution contained (in mM): 140 CsC1, 10 NaCl, 10 HEPES, 5 EGTA, 2 Mg ATP and 0.03 GTP, pH 7.3. The peak amplitudes measured either during the prepulse or the tail current (within 1 ms) were plotted as a function of the test potential and normalized to the maximal current obtained from the following Boltzmann function:

$${I}_{Tail} = \frac{{I}_{max} - {I}_{min}}{1 + exp ( ( V - {V}_{1 / 2} ) / s )} + {I}_{min}$$

Where V_{1/2} is the potential that elicits half maximal activation, s is the slope factor, and Iₘᵢₙ is the minimum current observed.

Oocyte electrophysiology. Defolliculated Xenopus laevis oocytes (harvested from adult females anesthetized with 0.5 g/l tricaine methanesulfonate) were injected with ˜10 ng of wild-type rat TRPV1 cRNA or mutant S502A/S800A TRPV1 cRNA (gift of Makoto Tominaga). Oocytes were placed in a Perspex chamber and continuously superfused (5 ml min-1) with Ca2+-free solution containing (in mM): 100 NaCl, 2.5 KCl, 5 HEPES, 1 MgCl2 and titrated to pH 7.3 with ˜5 mM NaOH. For solutions <pH 6.0, HEPES was replaced with either 5 mM MES or 5 mM sodium citrate. Oocytes were routinely voltage-clamped at −60 mV at 22-23° C. For heat activation, bath temperature was raised from ˜22-50° C. over ˜100 s using an in-line solution heater (Warner Instruments). The temperature was continuously monitored with a probe placed within 2 mm of the oocyte. The temperature-activation threshold was defined as a 20% increase in current above baseline.

Volatile general anesthetics. Saturated stock solutions of volatile general anesthetics (VGAs) were prepared in gas-tight bottles by dissolving excess anesthetic agents in bath solutions and stirring vigorously overnight. From these stock solutions fresh dilutions were made up every 40-60 minutes. Concentrations of volatile anesthetics in the bath solutions were verified using a modified head-space gas chromatography method. The gas chromatograph (Carlo Erba, Milan, Italy) was equipped with a flame ionization detector (FID) and mass spectrometer. The carrier gas was hydrogen (60 kPa column head pressure) and the fused silica capillary column, coated with polysiloxane SE-30, was 25 m×0.25 mm. Injector temperature was 250° C., FID temperature was 300° C. and the oven was maintained at 90° C. Standards were prepared from a mixture of halothane, isoflurane, and sevoflurane dissolved in acetonitrile with enflurane as an internal standard. The equivalent MAC were calculated using published conversion factors reported for halothane (1 MAC, 0.27 mM), isoflurane (1 MAC, 0.31 mM) and sevoflurane (1 MAC, 0.35 mM) in rat at 37° C. (Franks and Lieb, 1996).

Chemicals. Capsazepine, phorbol 12, 13 dibutyrate (PDBu), bradykinin and staurosporine were obtained from Sigma. Capsaicin and AMG9810 were purchased from Tocris Cookson (Ellisville, Mo.). Drugs were prepared as stock solutions in DMSO or ethanol and diluted into physiological solution prior to experiments. Statistical analysis. Data are given as mean±S.E.M. and statistical significance was evaluated using ANOVA or Student's t-test.

### Results

### Volatile Anesthetics Sensitize TRPV1 to Capsaicin and Protons

Although the data above showed that VGAs do not directly activate TRPV 1, it was considered that they could nonetheless sensitize TRPV 1 to other modes of activation. Indeed, a diverse array of physical and chemical stimuli activate TRPV 1 (Pingle et al., Handb. Exp. Pharmacol. 155-171 (2007)) and these stimuli produce synergistic effects when applied together. In sensory neurons, isoflurane (0.9 mM or ˜2.9 MAC) enhanced by approximately 3-fold whole-cell currents evoked by capsaicin (30 nM, FIG. 1A, n=5). Further, isoflurane increased capsaicin-evoked single channel activity in cell-free, outside out patches (FIG. 1B). It was observed that VGAs produced a similar sensitization of TRPV1 to protons. In TRPV1-expressing oocytes, isoflurane (0.9 mM) significantly enhanced by approximately 10-fold the currents evoked by a pH 5.5 solution (FIG. 2A, n=4). Dose-response analyses show that isoflurane reduced the half-maximal concentration required for activation by capsaicin and protons (FIGS. 1C&2B); the capsaicin EC50 was reduced from ˜1.6 to 0.8 μM (P<0.01) and the proton pEC50 was increased from 4.95 to 5.23 (P<0.01). In addition, isoflurane enhanced the maximal proton-evoked current by ˜3 fold.

### Anesthetics Enhance Voltage and Thermal Sensitivity of TRPV1

TRPV 1 is a voltage sensitive channel; membrane depolarization gates TRPV 1 and half-maximal activation (V1/2) is seen at ˜120 mV (at 25° C.) (Voets et al., Nature 430:748-54 (2004)). Although, these membrane potentials are supraphysiologic, agonists enhance the sensitivity of TRPV 1 to voltage such that the channel responds to voltage in the physiologic range. In addition, agonists increase the maximal voltage-evoked current (Matta and Ahern, J. Physiol. 585:469-482 (2007)). Similarly, it was observed that application of isoflurane (0.9 mM) enhanced the currents evoked by depolarization in HEK293 cells expressing TRPV1 (FIG. 3A). FIG. 3B shows the Boltzmann fits to these data. Isoflurane reduced the V1/2 by 23.0±6.2 mV and enhanced the maximal current by 15±6% (n=5).

TRPV1 is characteristically gated by heat with an activation threshold of ˜42-43° C. in mammalian cells and ˜46° C. in oocytes (Caterina et al., Nature 389:816-24 (1997)). It was determined whether isoflurane could alter this temperature sensitivity. In TRPV1-expressing oocytes isoflurane significantly reduced the temperature threshold in a dosedependent manner (FIGS. 3C&D); the thresholds for control, 0.5 mM and 0.9 mM isoflurane respectively were ˜46° C., 43° C. and 40° C.

### Clinical Concentrations of Diverse VGAs Regulate TRPV1

Next, it was determined whether VGAs could effectively modulate TRPV 1 at clinically-relevant concentrations. FIG. 4 shows that isoflurane (0.1 to 2 mM) enhanced proton-evoked responses in a dose-dependent manner and a significant potentiation occurred between 0.1 to 0.9 mM (corresponding to ˜0.3 to 3 MAC). Thus isoflurane, at concentrations achieved during maintenance anesthesia, is capable of enhancing TRPV 1 activity. Next, it was determined the effects of different VGAs. FIG. 5 show that VGAs (0.6 mM) with the most pungency, desflurane and enflurane, enhanced proton-evoked currents significantly more than the less pungent agents isoflurane and sevoflurane. Therefore, similar to the data presented above with TRPA1, there is a correlation, albeit less pronounced, between VGA pungency and TRPV 1 sensitization.

### PKC and Bradykinin Enhance VGA Activation of TRPV1

Many inflammatory mediators engage G-protein coupled receptors expressed on sensory neurons, leading to the activation of protein kinase C (PKC). In turn, PKC produces a marked sensitization of TRPV1 (Numazaki et al., J. Biol. Chem. 277:13375-13378 (2002); Premkumar and Ahern, Nature 408:985-90 (2000); Vellani et al., J. Physiol. 534:813-25 (2001)). It was observed that PKC significantly enhanced heat activation of TRPV1 by VGAs. After PDBu application, isoflurane (0.9 mM) reduced the temperature threshold further from 45.9±0.2 to 32.8±0.8° C., whereas PDBu alone reduced it to 39.3±2.3° C. (FIG. 3D). In contrast, PDBu did not produce a significant effect in oocytes expressing mutant TRPV 1 receptors that lack essential PKC-phosphorylation sites (S502A/S800A, 39.4±1.9° C. and 37.3±0.7° C. for isoflurane (n=5) and PDBu+isoflurane (n=3) respectively), indicating that PDBu produces its effects through direct phosphorylation of TRPV 1. This effect of PKC was more dramatic in mammalian cells. After PDBu treatment, VGAs (0.9 mM) evoked inward currents at room temperature (25° C.) in both TRPV1-expressing HEK293 cells (FIGS. 6A&C, n=7) and in capsaicin-sensitive sensory neurons (FIG. 6B-D, n=6). Further, isoflurane activated single TRPV1 channel activity in neurons after PDBu treatment. These responses were completely inhibited by the TRPV1-specific antagonist, AMG9810 (FIGS. 6B&D, n=3), indicating the selective activation of TRPV1.

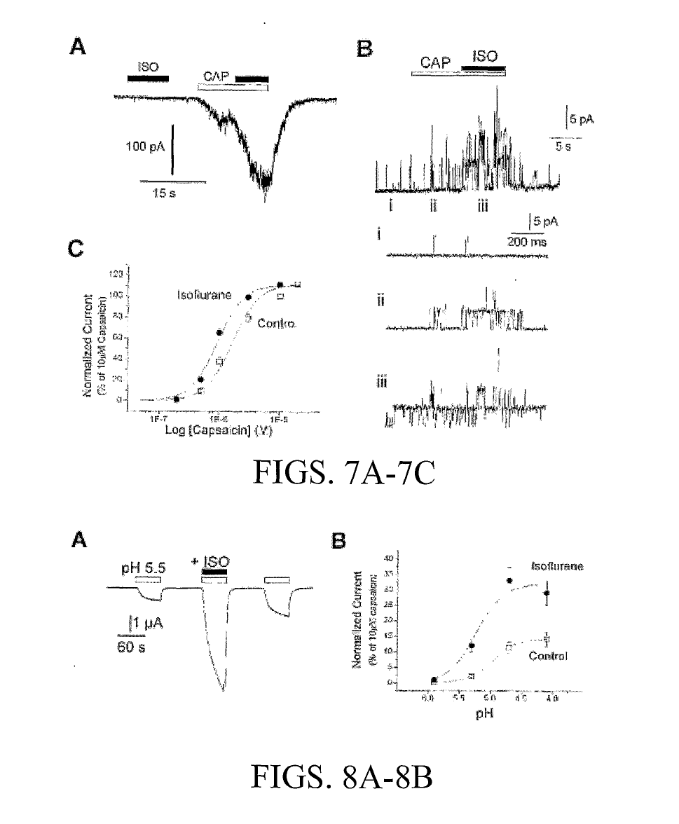

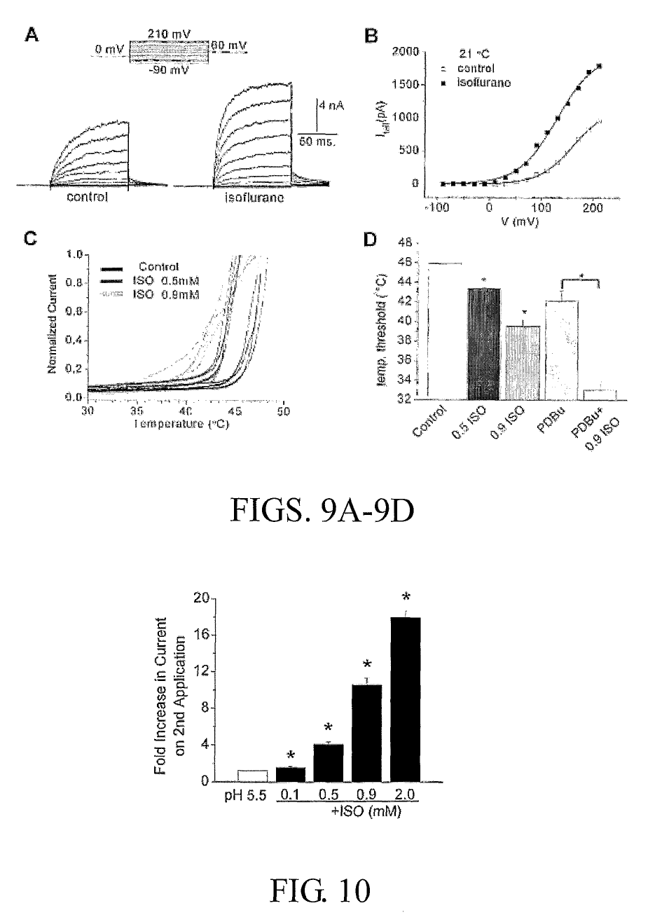

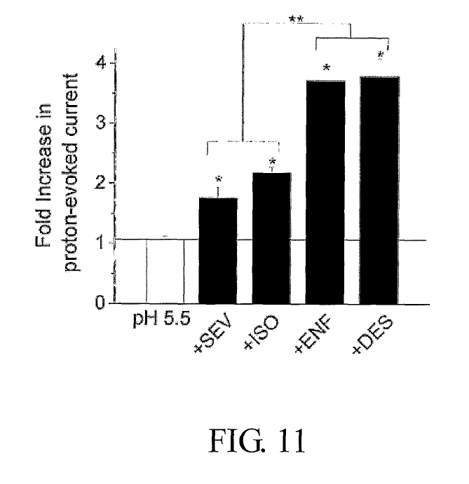

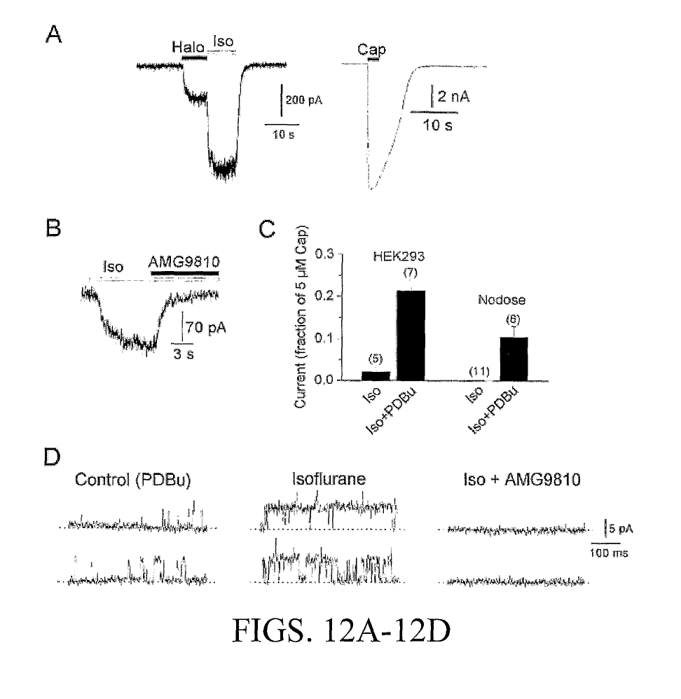

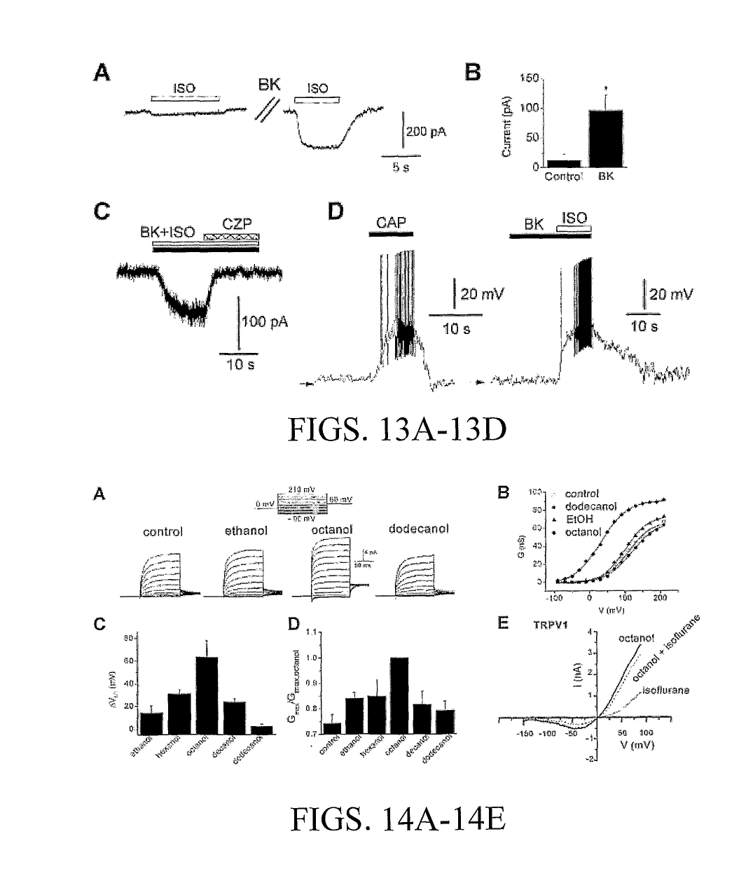

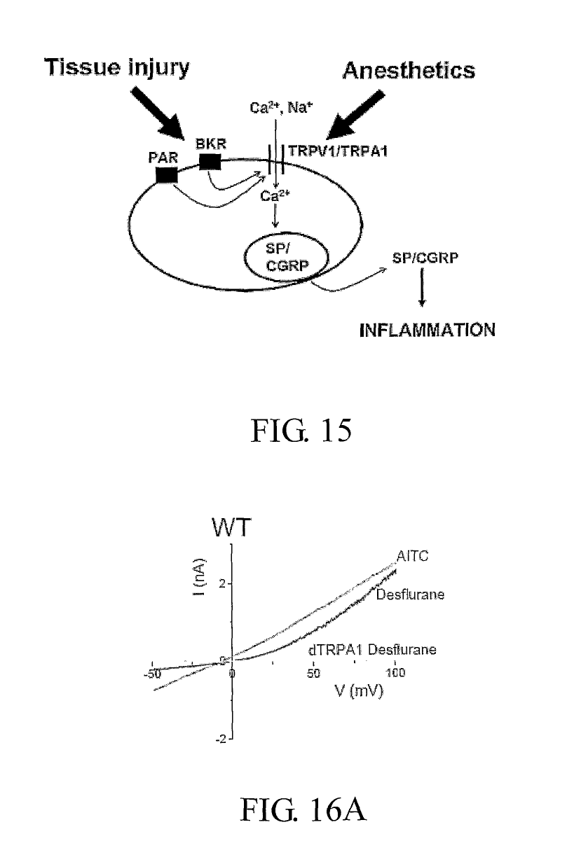

Surgery is associated with tissue injury and the release of numerous inflammatory mediators that can activate/sensitize sensory neurons. One key “pain” signaling molecule is bradykinin (BK). BK acting through its type two receptor can activate/sensitize TRPV1 (Cesare and McNaughton, PNAS 93:15435-9 (1996); Chuang et al., Nature 411:957-62 (2001); Premkumar and Ahern, Nature 408:985-90 (2000); Shin et al., PNAS 99:10150-5 (2002)) and TRPA1 (Bandell et al., Neuron 41:849-57 (2004); Bautista et al., Cell 124:1269-82 (2006)) via multiple signaling pathways. It was observed that BK enhanced the responses of sensory neurons to isoflurane (FIGS. 7C&D). Under control conditions isoflurane produced negligible responses, but after BK treatment, there was a marked increase in current (n=7, P<0.01). These neurons were all insensitive to AITC, excluding a contribution of TRPA1, and the TRPV1 blocker, capsazepine (1 μM), completely inhibited responses to isoflurane (in 3 of 3 cells), indicating that the major effect of BK was to recruit previously quiescent TRPV1 channels. These responses are sufficient to drive membrane excitability; under current-clamp, co-application of isoflurane and BK depolarized neurons and initiated sustained spike discharge (FIG. 7D, n=3). Taken together, these data provide strong support for the hypothesis that tissue injury can amplify the excitatory actions of VGAs on sensory neurons.

Volatile Anesthetics Interact Directly with TRPV1 Channels

VGAS could potentially alter TRPV 1 activity by altering cellular signaling cascades. However, the data presented herein showed that VGAs retained their effect on TRPV1 in cell-free patches indicating a membrane-delimited effect. To examine whether VGAs regulate TRPV 1 by directly interact with the TRPV 1 protein as opposed to effects on membrane fluidity, the action of long chain alcohols was investigated. The results of several studies indicate that VGAs and alcohols bind directly to GABAA and glycine receptors, at a common binding site located between transmembrane domains 2 & 3 (Mascia et al., PNAS 97:9305-10 (2000); Mihic et al., Nature 389:385-9 (1997)). Further, alcohols exhibit a carbon chain-length “cutoff”; the potency of alcohols increase with carbon chain length up until this cutoff, after which further increases in molecular size no longer increase alcohol potency (Mascia et al., PNAS 97:9305-10 (2000); Mihic et al., Nature 389:385-9 (1997)). These data are consistent with alcohols binding to a “pocket” on these channels of finite molecular volume. FIG. 8A-D shows that n-alcohols (2-12 carbons) enhanced voltage-dependent activation of TRPV1 in proportion to carbon chain length. Shifts in the V_{1/2} and increases in maximal conductance reached a maximum with octanol, thereafter, decanol produced a smaller response and dodecanol was without effect. Next, it was determined whether alcohols and VGAs act at similar binding site(s) on TRPV1. It was observed that isoflurane (0.9 mM) produced negligible effects on TRPV1 when applied together with an apparent saturating dose of octanol (1.8 mM) (FIG. 8E). This result was not due to a “ceiling effect” because the responses to octanol were submaximal (˜40% of that produced by 10 μM capsaicin at 200 mV). Therefore, these non-additive effects are consistent with VGAs and alcohols acting at the same site(s).

In summary, the data show that clinically-relevant concentrations of volatile anesthetics activate and sensitize the TRPV 1 channel. These results suggest that these VGAs may enhance peripheral nociceptive signaling in the context of surgery. The use of selective TRP antagonists will have utility by inhibiting the sensitizing effects of GAs as well as the generalized excitation of nociceptors by inflammatory mediators.

### Example 3

### Transmembrane Domain 5 of TRPA1 is Important for Desflurane Activation

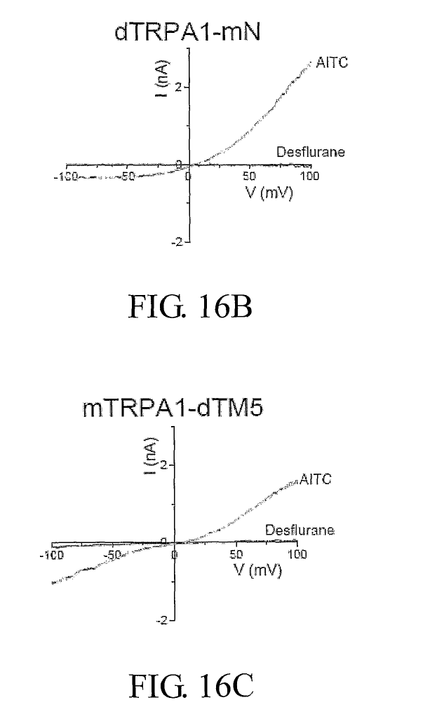

A genetic approach was used to identify critical sites in TRPA1 and TRPV1 required for activation by general anesthetics. Drosophila TRPA1 is insensitive to desflurane (at concentrations up to 3 mM). See FIGS. 16A, 16B and 16C, which shows that desflurane activates mouse TRPA1, whereas no responses are seen in cells expressing dTRPA1. In contrast, we found that dTRPA1 exhibited robust voltage-sensitivity indicating expression of functional channels. Note that dTRPA1 is insensitive to AITC. Next, several chimeric TRP proteins containing dTRPA1 (unresponsive to desflurane) and mTRPA1 were tested. For the dTRPA1-mN chimera, the mouse 720 amino acid N-terminus was exchanged for the drosophila N-terminus. The N-terminal domain contains essential binding sites for AITC, and therefore studying this chimera tests an important role for the N-terminus in anesthetic-sensing. FIG. 16B showed that while substituting the mouse N-terminus conferred AITC-sensitivity to dTRPA1, no responses to desflurane were evident. Thus, the N terminus does not appear to mediate sensitivity to volatile GAs. The mTRPA1-dTM5 construct was then studied, which is the mouse protein containing the fifth transmembrane domain of the drosophila protein. FIG. 16C showed that desflurane sensitivity was abolished in this chimera. Therefore these data suggest an important role for the TM5 domain in sensing volatile anesthetics. Table 1 shows the sequence alignment for the TM5 domain of dTRPA1 and mTRPA1.

TABLE 1
Sequence Alignment of TM5
Domain of dTRPA1 and mTRPA1.

| | |
|---|---|
| dTRPA1 TM5 | CLDFVGTYVNTYYRDQLKSVPMTSFLILS |
| domain | (SEQ ID NO: 1) |
| mTRPA1 TM5 | CGIFIGVMLEVIFKTLLRSTGVFIFLLLS |
| domain | (SEQ ID NO: 2) |

A number of aspects have been described. Nevertheless, it will be understood that various modifications may be made. Accordingly, other aspects are within the scope of the following claims.

1. A method for reducing or preventing inflammation in a subject comprising:
(a) selecting a subject in need of relief of inflammation; and
(b) administering to the subject an agent that inhibits the activity or expression of a transient receptor potential (TRP) ion channel.

2. The method of claim 1, wherein the inflammation in the subject is caused by an anesthetic.

3. A method for reducing or preventing pain in a subject comprising
(a) selecting a subject in need of relief of pain; and
(b) administering to the subject an agent that inhibits the activity or expression of a TRP ion channel.

4. The method of claim 2, wherein the pain is post-surgical pain.

5. The method of claim 2, wherein the pain in the subject is caused by an anesthetic.

6. The method of claim 1, wherein the subject is a surgical patient.

7. The method of claim 1, wherein the subject is under anesthesia.

8. The method of claim 1, wherein the agent is administered at the same time, before or after an anesthetic is administered to the subject.

9. The method of claim 1, wherein the agent inhibits the activity of TRP.

10. The method of claim 1, wherein the agent is selected from the group consisting of wortmannin, camphor, phosphatidylinositol-4,5-bisphosphate (PIP2), high levels of menthol, AP18, cannabinoids such as WIN 55,212-2, HC-030031, gadolinium, ruthenium red, capsazepine, AMG 517, SB366791, Iodo-resiniferatoxin, resiniferatoxin, LJO-328, and SC0030.

11. The method of claim 1, wherein the agent inhibits the expression of TRP

12. The method of claim 11, wherein the inhibitor of TRP expression is an inhibitory nucleic acid or small molecule.

13. The method of claim 12, wherein the inhibitory nucleic acid is selected from the group consisting of an antisense molecule, aptamer, ribozyme, triplex forming molecule, short interfering RNA (siRNA), and external guide sequence.

14. The method of claim 1, further comprising administering to the subject a second therapeutic agent.

15. The method of claim 14, wherein the second therapeutic agent is a pain medication or an anti-inflammatory agent.

16. The method of claim 1, wherein the TRP is transient receptor potential vanilloid (TRPV1) or TRP ankyrin (TRPA1).

17. The method of claim 1, wherein the agent binds the TM5 domain of TRPA1.

18. The method of claim 17, wherein the TM5 domain of TRPA1 comprises SEQ ID NO:1 or SEQ ID NO:2.


SEQUENCE LISTING (2 sequences)

SEQ ID NO: 1 - protein, 29 aa, Drosophila melanogaster
CLDFVGTYVNTYYRDQLKSVPMTSFLILS

SEQ ID NO: 2 - protein, 29 aa, Mus musculus
CGIFIGVMLEVIFKTLLRSTGVFIFLLLS